(19) United States
(12) Patent Application Publication
(10) Pub. No.: US 2024/0271146 A1
(43) Pub. Date: Aug. 15, 2024

(54) METHODS AND COMPOSITIONS OF MATTER FOR INERT BIOENGINEERING OF A BIOLOGICAL ENTITY

(71) Applicant: INDEX BIOSYSTEMS INC., Toronto (CA)

(72) Inventors: Michael BORG, St. Catharines (CA); Jeremy N. FRIEDBERG, Guelph (CA); David SINGER, Toronto (CA); Alessandra BIZ, York (CA)

(21) Appl. No.: 18/564,779

(22) PCT Filed: May 27, 2022
(86) PCT No.: PCT/CA2022/050865

§ 371 Date: Nov. 28, 2023

Related U.S. Application Data
(60) Provisional application No. 63/194,761, filed on May 28, 2021.

(51) Int. Cl. C12N 15/81; C12N 15/90

(52) U.S. Cl. CPC C12N 15/81; C12N 15/905; C12N 2310/20

(57) ABSTRACT

A bioengineering method which comprises introducing an inert nucleic acid cassette into a biological entity without introducing or modifying characteristics or traits in the biological entity. The method comprises receiving or providing a sample comprising the biological entity having a nucleic acid sequence; selecting an integration site in the nucleic acid sequence for inserting the inert nucleic acid cassette; designing the inert cassette with optimized primer sequences, optimized probe sequences, optimized stop codons and disrupted start codons, and inserting the inert nucleic acid cassette into the biological entity at the integration site; and validating that no characteristics have been added or modified in the biological entity.

### FIELD OF THE INVENTION

The present invention generally relates to the identification and/or tracking of supply chain products and materials. More specifically, the present invention relates to methods and compositions for the inert bioengineering of a biological entity meant to be used as a proxy for tracking supply chain products and materials.

### BACKGROUND OF THE INVENTION

The concept of biological identification using DNA barcodes which are native to an organism was first developed by Dr. Hebert at the University of Guelph. These barcodes consist of sequences of nucleotides unique to the organism or biological entity. These breakthrough technologies made it is possible to efficiently detect DNA barcodes in isolated DNA using standard DNA amplification and molecular biology protocols. These barcoding techniques quickly became ubiquitous in various industries and are commonly used in environmental DNA research; and within commercial inspection, verification, testing and certification companies.

A decade after Hebert published his paper in 2003, two patent applications (US 2019/0285602 and US 2017/0021611) were filed relating to the use of synthesized cell-free DNA markers for tagging products throughout supply chains. These DNA markers rely on industrial-scale polymerase chain reaction (PCR) manufacturing systems, and remain prohibitively expensive for large-scale applications. In addition, extracellular or cell-free DNA is unstable when exposed to the extracellular environment, or when added to industrial processes. This instability has prompted research in encapsulation techniques meant to protect the integrity of cell-free DNA fragments. One example uses silica to encapsulate DNA-based tracers. Encapsulated DNA-based tracer solutions continue to have substantial economic limitations relative to standard cell-free DNA, which makes them unsuitable for scalable industrial supply chain adoption.

Thankfully, advancements in biotechnology have created efficiencies such that it is now economically feasible to edit the genomes of almost any organism. Moreover, it is now feasible to develop biologically traceable technologies that leverage the replicative and protective capacity of an organism to identify any biological entity in the supply chain, and/or to function as a proxy for the identification of supply chain products and materials. These technological breakthroughs enabled DNA-based biomarking without relying on expensive industrial PCR or encapsulation approaches. Index Biosystems bioengineered short identifying barcode sequences of DNA into an organism and scale using industrial fermentation bioaccumulation. However, this technology can result in potentially restrictive regulatory treatment since it relies on genetically engineering an organism. As such, it becomes essential to address concerns of risk associated with the bioengineering of the host organism. There is thus need for methods and compositions of matter which enable the introduction of exogenous DNA and/or RNA into a recipient organism or biological entity such that no characteristics are introduced, and no existing characteristics are modified.

### SUMMARY OF THE INVENTION

The shortcomings of the prior art are generally mitigated by the method as described herein for inert bioengineering of a biological entity, which involves introducing an inert nucleic acid cassette into the biological entity without introducing or modifying characteristics or traits in the biological entity.

Accordingly, there is provided herein a method which comprises receiving or providing a sample comprising the biological entity having a nucleic acid sequence. An integration site is selected in the nucleic acid sequence for inserting the inert nucleic acid cassette. Once the site has been selected, the method then comprises designing the inert cassette with optimized primer sequences, optimized probe sequences, optimized stop codons and disrupted start codons, and inserting the inert nucleic acid cassette into the biological entity. The following step comprises validating that no characteristics have been added or modified in the biological entity. Optionally, the method may include a step of inactivating the biological entity.

According to a preferred embodiment, selecting an integration site comprises determining if a new site is required. For example, this may include checking a data store with information on the biological material to ascertain if a prior engineering event has occurred, and thus if a new site is required.

According to a further embodiment, selecting an integration site comprises screening the genome of the biological entity and identifying any characterized and/or putative genes, open reading frames and/or features of concern.

According to a further embodiment, the selected integration site is within a predetermined distance in base pairs (bp), preferably at least 250 base pairs (bp), from putative genes, open reading frames and/or features of concern. In a non-limiting embodiment, the predetermined distance is typically between 250-1000 bp from the putative genes, open reading frames and/or features of concern.

According to a further embodiment, the selected integration site is located within heterochromatic regions and/or long terminal repeats (LTRs).

According to a further embodiment, selecting an integration site comprises determining an absence of characterized and/or putative promoters directing transcription of the cassette.

According to a further embodiment, designing the inert cassette comprises creating landing pads for assisting with the integration of the inert cassette at the integration site.

According to a further embodiment, the landing pad is a CRISPR-Cas9 target sequence complementary to an optimized gRNA spacer.

According to a further embodiment, the integration site is selected in silico using databases.

According to a further embodiment, inactivating the biological entity comprises thermal inactivation, chemical inactivation, nutrient deprivation over time, or a combination thereof.

In further non-limiting embodiments, the step of validating that no characteristics have been added or modified in the biological entity may include analyzing the transcriptional profile of the biological entity to validate that transcripts of the inert nucleic acid cassette cannot be detected

In another non-limiting embodiment, the step of validating that no characteristics have been added or modified in the biological entity may include whole genome sequencing of the transformed biological entity to validate that the inert nucleic acid cassette has been integrated at the selected site, without any off-target effects. Without limitation, off-target effects may include one or more mutations in the cassette or elsewhere in the genome of the biological entity. Also provided herein is an inert nucleic acid cassette for inert bioengineering of a biological entity, the inert nucleic acid cassette comprising optimized primer sequences, optimized probe sequences, optimized stop codons and disrupted start codons.

In an embodiment of the inert nucleic acid cassette, the optimized stop codons may comprise stop codons for all reading frames in order to stop any transcription initiation within the cassette and the sequences flanking the cassette.

Other and further aspects and advantages of the described method will be better understood upon the reading of the illustrative embodiments about to be described or will be indicated in the appended claims, and various advantages not referred to herein will occur to one skilled in the art upon employment of the invention in practice.

### BRIEF DESCRIPTION OF THE DRAWINGS

The aspects, features and advantages of the invention will become more readily apparent from the following description, reference being made to the accompanying drawings in which:

FIG. 1 is a flow chart of a method for inert bioengineering of a biological entity.

FIG. 2 is a flow chart of a method for selecting an appropriate integration site, according to an embodiment.

FIG. 3 is an example of a selected site, according to an embodiment.

FIG. 4 is a flow chart of a method for designing the inert cassette, according to an embodiment.

FIG. 5 is an example of an inert cassette design, according to an embodiment.

FIG. 6 is a flow chart of a method for validating that no characteristics have been added or modified in the recipient organism, according to an embodiment.

FIG. 7 is an example of an RNA transcription validation step, according to an embodiment.

FIG. 8 is an example of growth validation step, according to an embodiment.

FIG. 9 is an example of a sugar consumption validation step, according to an embodiment.

FIG. 10 is a flow chart of a method for inactivating the recipient organism according to an embodiment.

FIG. 11 is an example of thermal inactivation that is validated using trypan blue staining according to an embodiment.

FIG. 12 is an example of an inert cassette design, according to a further embodiment.

FIG. 13 is an example of a selected site, according to an embodiment.

FIG. 14 is an example of a selected site, according to an embodiment.

FIG. 15 is an example of a selected site, according to an embodiment.

FIG. 16 is an example of an RNA transcription validation step, according to an embodiment.

FIG. 17 is an example of growth validation step, according to an embodiment.

FIG. 18 is an example of a sugar consumption validation step, according to an embodiment.

FIG. 19 is an example of an RNA transcription validation step, according to an embodiment.

FIG. 20 is an example of growth validation step, according to an embodiment.

FIG. 21 is an example of a sugar consumption validation step, according to an embodiment.

FIG. 22 is an example of an RNA transcription validation step, according to an embodiment.

FIG. 23 is an example of growth validation step, according to an embodiment.

FIG. 24 is an example of a sugar consumption validation step, according to an embodiment.

### DETAILED DESCRIPTION

Described herein are methods and compositions for inert bioengineering of a biological entity. It will be appreciated that embodiments and examples are provided for illustrative purposes intended for those skilled in the art, and are not meant to be limiting in any way.

The principles and methodologies for assessing the risk of a modified organism has been internationally ratified under the Cartagena Protocol. The Cartagena Protocol on Biosafety is an international agreement which aims to ensure the safe handling, transport and use of living modified organisms (LMOs) resulting from modern biotechnology that may have adverse effects on biological diversity, taking also into account risks to human health.

It reasonably follows that the design of these barcodes and the subsequent bioengineering events must address risks associated with the points-to-consider within the Cartagena Protocol. The points-to-consider described by the protocol consist of:
Recipient organism or parental organisms
Donor organism or organisms
Vector
Insert or inserts and/or characteristics of modification
Living modified organism
Detection and identification of the living modified organism
Information relating to the intended use
Receiving environment

The common thread throughout these points-to-consider relate to the introduction of characteristics or the modification of existing characteristics. If one were to, in absolute terms, eliminate risks associated with these points-to-consider it would be achieved by bioengineering an inert cassette into a target site within the recipient organism or biological entity such that no characteristics were introduced and no characteristics were modified.

The terminology used herein is in accordance with definitions set out below.

As used herein the term “biological entity” refers to any organism such as an individual animal, plant, or single-celled life form and microorganisms such as bacteria, yeast, viruses, and fungi.

As used herein the term “characteristics” comprises any expressed traits or attributes of the organism original to the organism and prior to the integration of the inert nucleic acid cassette in the genome of the organism. In this context, biological characteristics include but are not limited to metabolic rates and profile; rate of growth, development and reproduction; lipids, carbohydrates and proteomic profiles; sensitivity or response to the environment and structural organization and homeostasis.

As used herein the term “site” is used to refer to a location, or locus in the genome or nucleic acid sequence of a biological entity where the cassette will be integrated.

As used herein the term “landing pads” is used to refer to exogenous sequences introduced in the genome or nucleic acid sequence of the biological entity to assist with the integration of the cassette at the selected integration sites.

As used herein the term “cassette” is used to refer to a nucleic acid sequence containing optimized primer and probe sequences and stop codons, and disrupted start codons.

As used herein the term “nucleic acid sequence” is used to refer to all genetic material of an organism including chromosomal DNA and extra-chromosomal nucleic acid such as DNA plasmids, mitochondrial DNA and viral RNA. In certain embodiments, “nucleic acid sequence” will refer to the genomic DNA or RNA sequence of the biological entity.

As used herein the expression “predetermined threshold” is used to refer to the threshold at which the difference between the expression levels of the inert cassette relative to the wild-type becomes statistically significant.

By “about”, it is meant that the value or the number of nucleic acid can vary by 10% of the recited value.

Provided herein are methods and compositions for inert bioengineering of a biological entity, such that a traceable nucleic acid is introduced into the nucleic acid sequence of the biological entity without introducing or modifying characteristics or traits. In certain embodiments, methods as described herein may make use of a unique inert identifier sequence (also referred to herein as a nucleic acid unique identifier sequence), exogenously introduced (i.e. inserted/integrated) into the genome or nucleic acid sequence of a biological entity, in order to provide for identification and/or traceability of the biological entity itself, and/or materials comprising the biological entity and/or materials produced from the biological entity and containing genomic DNA or RNA therefrom. In certain embodiments, strategies as described herein may benefit from the durability and replicative capacity of nucleic acid such as DNA or RNA to provide identification and/or traceability. Accordingly, the traceability of the materials may in certain non-limiting applications be utilized in the context of authentication and/or identification of biological materials within supply chains. For example, tracking supply chain products and materials within the following industries are contemplated but not limited thereto: fluid management for oil and gas, electronics, packaging, textiles, mining, food, animal feed, pharmaceuticals, and nutraceuticals which includes biologics, synthetics and supplements. Other applications will be apparent to those of ordinary skill in the art.

### Example 1

### Methods for Inertly Bioengineering a Biological Entity

According to a preferred embodiment, there is provided a method for bioengineering a biological entity by introducing exogenous nucleic acids comprising an inert cassette into the biological entity without introducing or modifying characteristics in the biological entity. The method comprises receiving or providing a sample comprising the biological entity having a nucleic acid sequence; selecting an appropriate integration site in the nucleic acid sequence for insertion of the inert cassette; designing the inert cassette comprising optimized primer sequences, probe sequences and stop codons, and disrupted start codons, and inserting the inert nucleic acid cassette into the biological entity at the selected integration site; validating that no characteristics have been added or modified in the biological entity; and inactivating the biological entity.

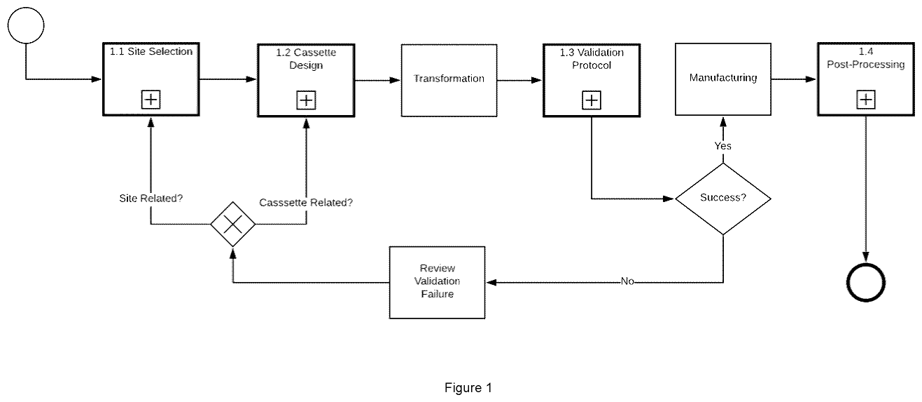

A summary flowchart of the method for inertly identifying a biological entity according to a preferred embodiment is depicted in FIG. 1.

### Selection of an Integration Site

The method first comprises receiving or providing a sample comprising the biological entity. The nucleic acid sequence of the biological entity instructs the selection of an appropriate integration site in the nucleic acid sequence for insertion of an inert cassette. Integration sites may have already been established for any given biological entity. As such, the selection step first entails determining if a new site is required using one or more databases well known the art. If a new site is required, the properties for a suitable site may be determined experimentally using a variety of molecular biology approaches well known in the art since these properties may vary from organism to organism, or strain to strain. After having determined the genetic landscape and considered a plurality of properties of the biological entity, a selection of a target site for insertion of an inert cassette is made within the constraints of the available genomic data.

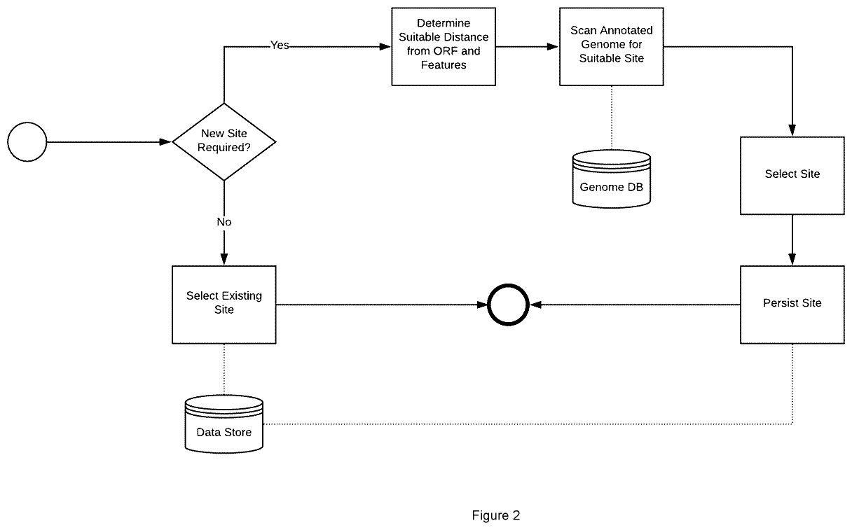

A summary flowchart of the method for selecting an appropriate integration site according to a preferred embodiment is depicted in FIG. 2.

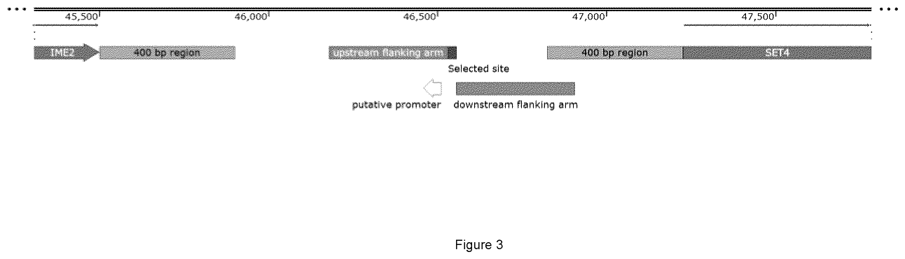

The selection of an integration site within Saccharomyces cerevisiae is depicted in FIG. 3. The selection first entails screening the host genome and identifying any characterized, putative genes, open reading frames and/or features of concern. Potential sites within a predetermined distance (measured in nucleotide base pairs, denoted bp) from characterized, putative genes, open reading frames and/or features of concern, in any direction on either nucleic acid strand are then selected. One criterion that may be used to identify potential sites is a distance of at least 250 bp since this distance is expected to be sufficient to avoid the disruption of the proximal promoter regions, which in eukaryotes are about 250 bp away from genes (Goni et al., 2007). The distance of at least 250 bp is also expected to be sufficient to avoid the disruption of the 5′ UTR in terminators, which typically ranges 100-200 bp away from stop codons across several life domains (Mignone et al., 2002). Another criterion which may be used to select the site is the absence of characterized and/or putative promoters directing to the transcription of the cassette, which may be screened in silico using, for example, methods described in Reese (2001). FIG. 3 illustrates a suitable site given the desired properties described hereinabove.

In addition, the selected sites may preferably be located within heterochromatic regions which are loci typically found near telomeres and centromeres that are characterized by tightly packed DNA. The position of heterochromatic genes in these regions is known to cause gene silencing (Gartenberg and Smith, 2016). It may be desired to integrate the cassette within heterochromatic regions in order to achieve silencing. Furthermore, the selection of sites may preferably be located within Long Terminal Repeats (LTRs) which are repeated regions within the genome that are reminiscent of a retroviral infection (Coffin et al., 1997). In addition to not disrupting critical genes native to the host organism, the integration of the cassette in LTR regions can have the added benefit of creating extra copies of the cassette, as these loci are often repeated in the genome dozens or even hundreds of times (Shi et al., 2016).

### Design of the Inert Cassette

Once the target site has been selected, the method follows with the design of an inert cassette having an arbitrary sequence for integration in the biological entity. In order to design an appropriate inert cassette without introducing or modifying characteristics in the biological entity, a stop codon configuration is selected from one or more host codon-optimized databases well known in the art. These stop codons may be variably distanced from each other and repeated throughout the cassette in pre-determined combinations. Once selected, the stop codon configuration is added to the cassette, which is then reviewed for any additional sequences of concern in view of the wild-type biological entity and databases of known nucleic acid motifs. The identified sequence(s) of concern are then disrupted until there are no remaining sequences of concern. In addition, any start codons that arise from the design will be disrupted. The resulting cassette's sequence is then stored in a database.

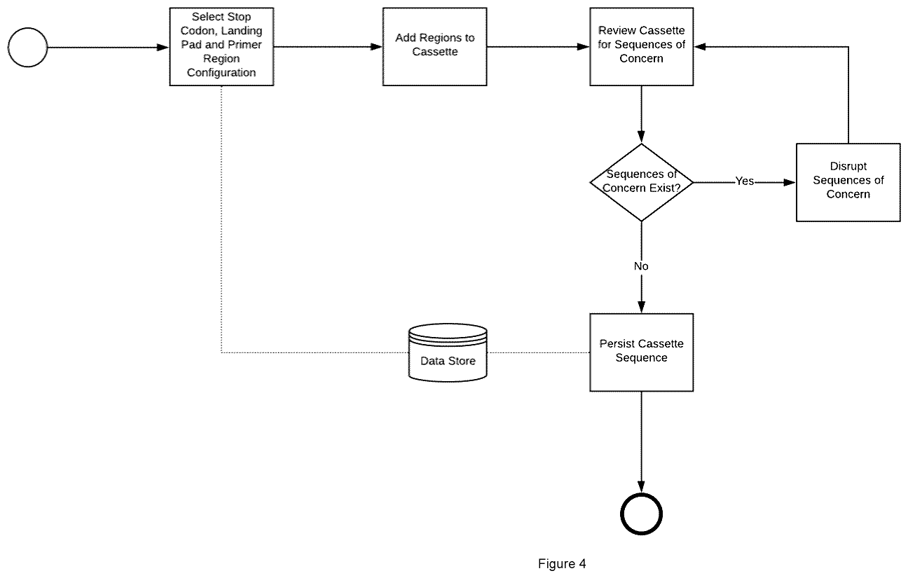

A summary flowchart of the method for designing the inert cassette, according to a preferred embodiment is depicted in FIG. 4.

An example of a cassette design (SEQ ID NO: 1) is depicted in FIG. 5. The cassette may be up to ˜1500 bp long and may contain optimized primer sequences designed in silico, reproduced from the literature, or selected within the cassette for qPCR (SEQ ID NOs: 3 and 12) (Bustin and Huggett, 2017), sequencing (Dieffenbach et al., 1993), LAMP assays (SEQ ID NOs: 4, 5, 7, 9, 10, and 11) (Jia et al., 2019), RPA (Higgings et al., 2018), and/or other amplification techniques. The cassette may also contain sequences optimized in silico for qPCR probes (SEQ ID NO: 8) (Bustin and Huggett, 2017), or sites for gRNA binding for use with CRISPR-Cas editing methods (Bourgeois et al., 2018). The cassette should contain stop codons for all of the 6 reading frames and in both the 3′ and 5′ ends of the cassette (SEQ ID NOs: 2 and 13) as well as on both sense and antisense strands, in order to stop any transcription initiation within the cassette and the sequences flanking the cassette. For large cassette designs that are, for example, larger than 100 bp, stop codon sequences for all reading frames will be randomly inserted in-between every 50-300 bp, as depicted in FIG. 5 (SEQ ID NOs: 2, 6 and 13). This stop codon design will mitigate risks associated with any previously undetected, low levels of transcribed RNA resulting in a functional protein product. A cut-off size of about 300 bp, or 100 amino acids, is typically the threshold for the annotation of open reading frames (ORFs) because peptides that are smaller than 100 amino acids are unlikely to have stability, folding capacity and/or biochemical activity (Dujon et al., 1994; White, 1994). For example, a 6 frame stop codon sequence may be TTAATTAATTAA. The cassette is designed such that start codons are removed or disrupted. In addition, the designed nucleic acid sequence of the inert cassette is screened in silico for absence of putative promoters (Reese, 2001) and other motifs of concern. If putative promoters or concerning motifs exist, the cassette is redesigned.

Integration of the cassette may be conducted with a CRISPR-Cas assisted double strand break followed by a homology-directed repair (DiCarlo et al., 2013). In this approach, the Cas nuclease is expressed in a species-specific high copy plasmid between a suitable species-specific promoter and terminator and selected by a dominant marker, for example, an antibiotic resistance gene. In addition, the plasmid contains a species-specific RNA polIII promoter to drive the expression of the gRNA compatible with the designated Cas, in which the spacer was selected to target the selected site. Other methods for integrating the inert nucleic acid cassette may be used while remaining within the scope of the present invention. The cassette may be produced as a synthetic nucleic acid block including homology arms of 75-500 bp that direct the nucleic acid repair and cassette integration within the site. A suitable nucleic acid transformation is performed and should be adapted for each organism. Thereafter, the transformed organisms are pre-screened for the presence of the nucleic acid sequence of the cassette at the desired site, a process sometimes called colony-PCR, a method that is described for example in Sheu et al., 2000. Other methods for pre-screening for the presence of the nucleic acid sequence of the cassette at the desired site may be used while remaining within the scope of the present invention.

Once pre-screen is completed, a positive organism is submitted to culturing in the absence of the plasmid selection, for example, in the absence of antibiotic, to remove the nuclease expression plasmid, a process that sometimes is called curing of the plasmid, as described by Rodríguez-López et al., 2017. After the strain is cured of nuclease plasmid, a series of validations can follow concurrently.

### Validation of the Inert Cassette

Once the cassette has been designed in silico, the cassette can then be synthesized experimentally and inserted into an appropriate plasmid using standard molecular biology techniques well known in the art. The cassette-containing plasmid is then transformed into the biological entity for validation steps to ensure that the transformed biological entity meets the requirements for an inert bioengineering event. The genomic nucleic acid of the biological entity is first extracted and submitted to whole genome sequencing (WGS), to assess if the cassette has the correct sequence, to confirm that it was integrated in the correct site, and to verify if any notable validation errors may have occurred. These validation errors include but are not limited to 1) Off-target effects review 2) Integration site review and 3) Cassette sequence review processes. If an off-target mutation event occurs, another colony can be screened. This can be repeated several times until a selected colony meets the validation criteria. If validation fails after a certain number of iterations, a redesign of the inert cassette may be required. It is not necessary that all of these validation steps be completed in each validation process run. The bioengineering validation steps are critical to ensure that the transformant meets the requirements for an inert bioengineering event. Additional validation steps may be taken or some validation steps may be omitted while remaining within the scope of the present invention. The complex gateway denoted by the [symbol] icons describe tasks within a process that may vary. However, where some set of tasks are required, that whole set must be completed successfully. As soon as a single task within that set fails, the whole process fails and terminates. In both cases (success and failure) the results are recorded in a database.

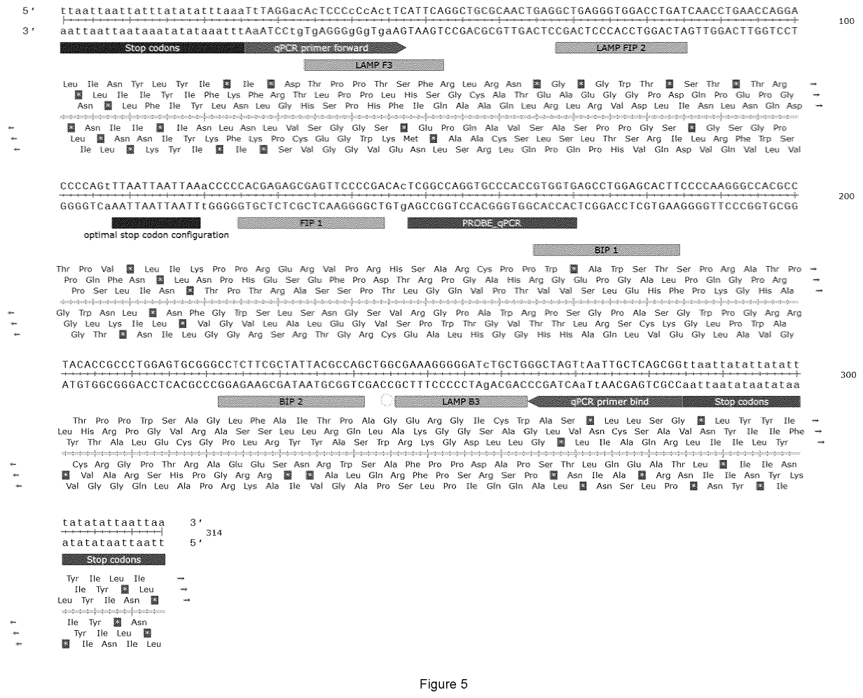

A summary flowchart of the method for validating that no characteristics have been added or modified in the biological entity, according to a preferred embodiment is depicted in FIG. 6.

In an exemplary embodiment, Saccharomyces cerevisiae S288C was transformed with an integration cassette of 370 bp in a site 810 bp distant of the gene TRM7 and 1461 bp distant from the gene OCR2 using CRISPR-Cas9 and gRNA expression plasmids with a hygromycin resistance gene. The pre-screen colony-PCR was carried out by extracting the genomic DNA of 4 transformants and carrying out colony PCR with primers amplifying a region corresponding to 500 bp flanking the integration site in the wild-type strain. Agarose gel electrophoresis showed an increase in molecular weight of the DNA fragment corresponding to the inserted inert cassette. This indicated that the transformants had successfully integrated with the cassette in the selected site.

Validation of the genomic integration of the inert cassette into the biological entity is followed by a phenotypical characterization to validate that the integration of the cassette did not introduce any characteristics or modify the characteristics of the biological entity.

Accordingly, the biological entity is cultured under biomass production conditions and the RNA extracted for expression review. For example, expression review can be based on reverse transcriptase qPCR targeting the amplification of the cassette for quantifying the expression level of the cassette, (as described in Freeman et al., 1999) compared to the wild type strain without the cassette. If the signal is more than what is identified as the acceptable upper threshold, the site and/or the cassette may need to be redesigned. Other methods for expression review may be used while remaining within the scope of the present invention.

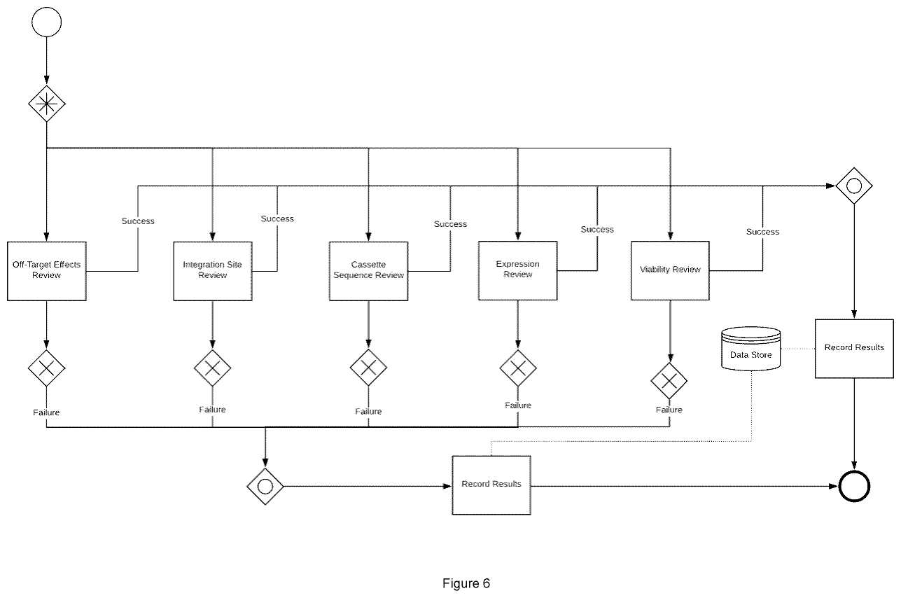

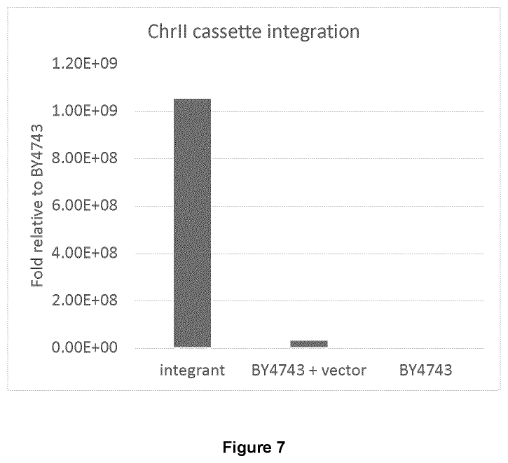

In the example depicted in FIG. 7, one of the pre-screened Saccharomyces cerevisiae S288C transformants was cultured in the biomass production conditions and its RNA was extracted (integrant), alongside with a strain where the same 370 bp cassette was cloned to a plasmid (BY4743+vector), and the wild type strain (BY4743). The RNA was reverse transcribed to cDNA, which then was submitted to qPCR using primers and probes that bind within the cassette. The integrant shows a significant fold increase in the expression of the cassette compared to what was observed from the cassette being incorporated in a plasmid (BY4743+vector), or in the absence of the cassette (BY4743). This clearly show that unforeseen RNA expression can occur depending on the combination of site/cassette DNA sequence, for example, in the insertion of the cassette downstream of an uncharacterized promoter.

The phenotypical characterization follows with a viability analysis. The biological entity comprising the inert cassette is cultured alongside and in the same conditions as the wild-type strain to assess if the growth curves overlap to determine whether the integration of the cassette perturbed any genes, metabolic regulation or any other features that might regulate growth.

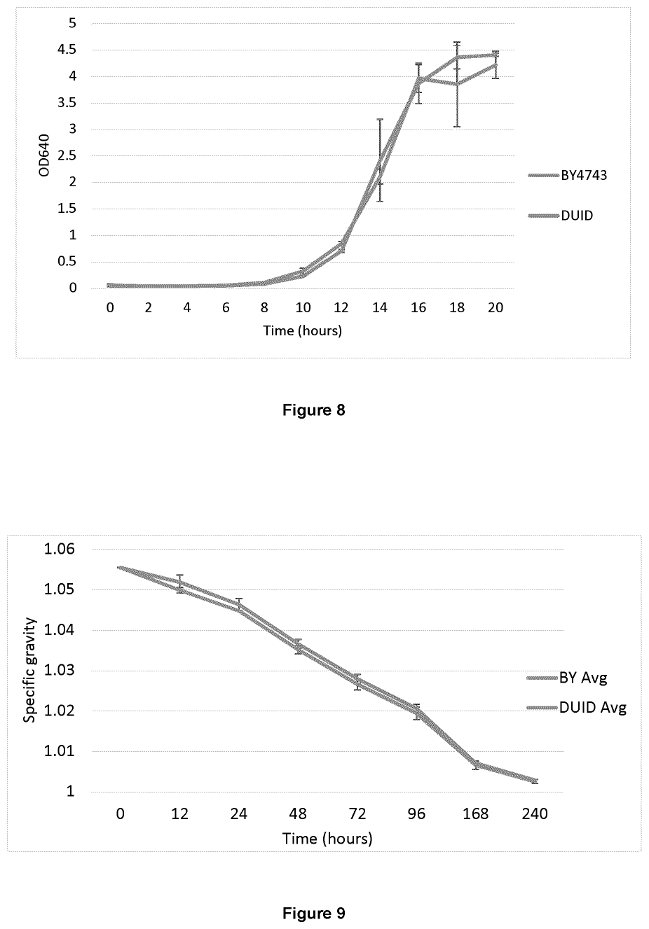

In the example depicted in FIG. 8, the growth curves of the wild type Saccharomyces cerevisiae S288C strain (green), and integrant (blue) overlap, as measured by the OD640 over time. This indicates that the introduction of the cassette did not introduce any characteristics that negatively affect growth.

In addition, metabolic regulation may be assessed by comparing the sugar consumption profile of the biological entity containing the cassette to the wild type strain, as depicted in FIG. 9. In this example, the sugar consumption was followed using specific gravimetry (De Clerck, 1958). This can be carried out in microplates with automated plate reader or by manually taking samples overtime and accessing the change in the optical density signal, which correlates with biomass concentration, as described in Sonderegger and Sauer (2003). The curves of sugar consumption of the wild type Saccharomyces cerevisiae S288C strain (green), and integrant (blue) overlap, as measured by specific gravity. This indicates that the introduction of the cassette did not introduce any characteristics that negatively affect sugar metabolism. It is to be understood that other biological characteristics well known the art may be evaluated to validate that no characteristics have been added or modified while still remaining within the scope of the present invention. For example, molecular and cellular signatures, sensitivity or response to the environment, reproduction, growth and development, regulation, homeostasis, and energy processing may be assessed.

The validated biological entity can then qualify for commercialization and stored in the strain repository. Alternatively, further validation steps may be undertaken to satisfy regulatory hurdles and/or to ensure that no characteristics have been modified or added to the biological entity. These further validation steps may include chemical equivalency and proteomics with mass spectrometry, for example.

### Post-Processing

According to a preferred embodiment, further post-processing steps are included to limit the potential for generational mutations and environmental release. Post-processing comprises inactivating the biological entity, followed by growth experiments which are well known in the art to ensure the inactivation was successful. The inactivation steps for a host organism is selected and adapted according to the selected species. A thermal inactivation (as in Couto et al., 2005), a chemical inactivation (Li and Wu, 2013), or a combination of these two processes may be carried out. For example, thermal inactivation can be accomplished by incubation of the fermentation broth containing the biomass, or biomass washed with water or buffer, at a certain temperature, for example 85° C., for a certain amount time, for example 20 minutes. Chemical inactivation can be accomplished by the addition of acetic acid to the fermentation broth containing the biomass, or biomass washed with water or buffer, to a certain concentration, for example, 5-1% v/v, letting it sit for a certain period of time in treatment, for example, 20 minutes. Cell inactivation can be followed by cell staining methods, such as trypan blue, which gives an indication of the extent of cellular death and as such indicates if the method was effective (Kucsera et al., 2000).

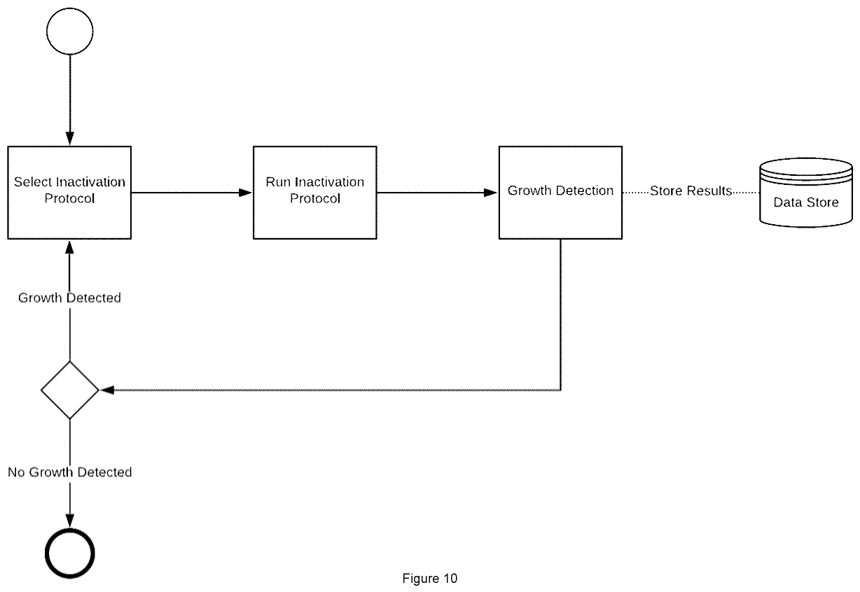

A summary flowchart of the method for inactivating the biological entity, according to a preferred embodiment is depicted in FIG. 10.

In the example depicted in FIG. 11, trypan blue staining was selected to assess cell death. The selected organism, Saccharomyces cerevisiae S288C, was incubated at 85° C. for 20 min. Cells stained with trypan blue and showing positive blue staining indicate cell death, which may in turn indicate that the treatment was effective, although further confirmation with CFU/mg would be required to make a definitive conclusion.

### Example 2

### Inert Cassette Design & Characterization

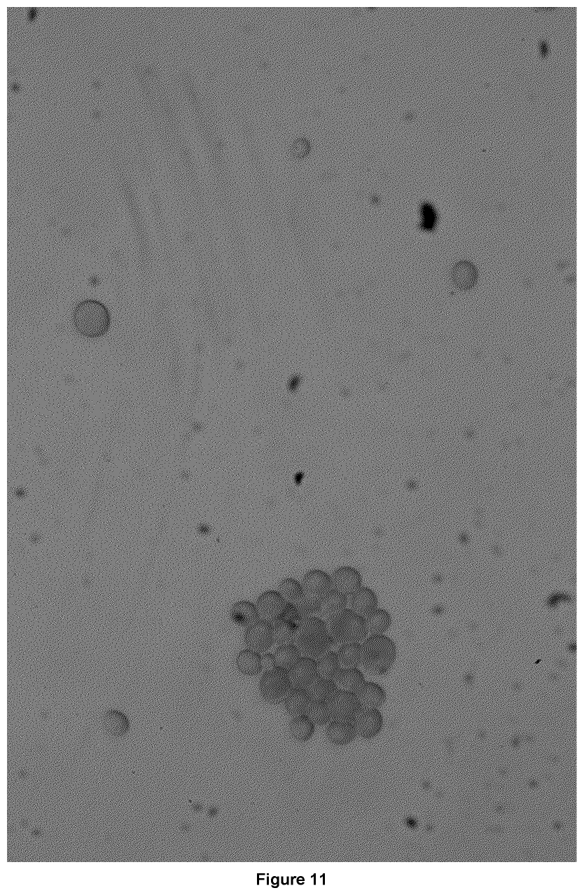

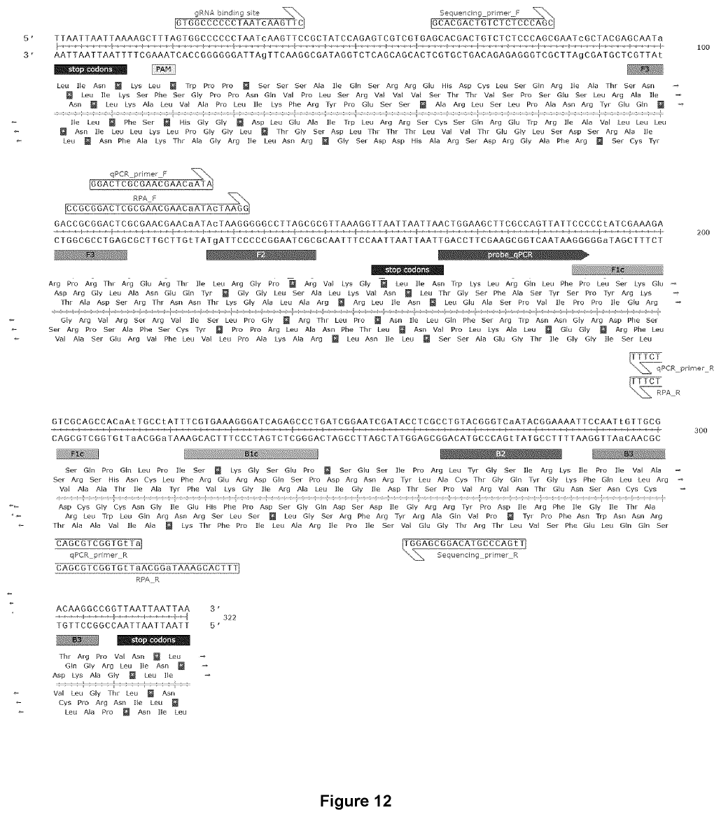

Another example of an inert cassette design (SEQ ID NO: 14) is depicted in FIG. 12. The cassette is 322 bp long and contains primers designed in silico for sequencing (SEQ ID NOs: 18 and 29), qPCR (SEQ ID NOs: 21 and 27), loop mediated isothermal amplification (LAMP) (SEQ ID NOs: 19, 22, 25, 28, 30 and 31), and recombinase polymerase amplification (RPA) (SEQ ID NOs: 20 and 26). The cassette also contains a qPCR probe (SEQ ID NO: 24), and gRNA binding sequence (SEQ ID NO: 16 and 17). The cassette also contains strategically distributed stop codons for all the 6 reading frames (TTAATTAATTAA). These stop codons are in both flanks of the cassette as well as within the cassette (SEQ ID NOs: 15, 23 and 32). The inert cassette was screened for putative promoter sequences with the method described in Reese (2001).

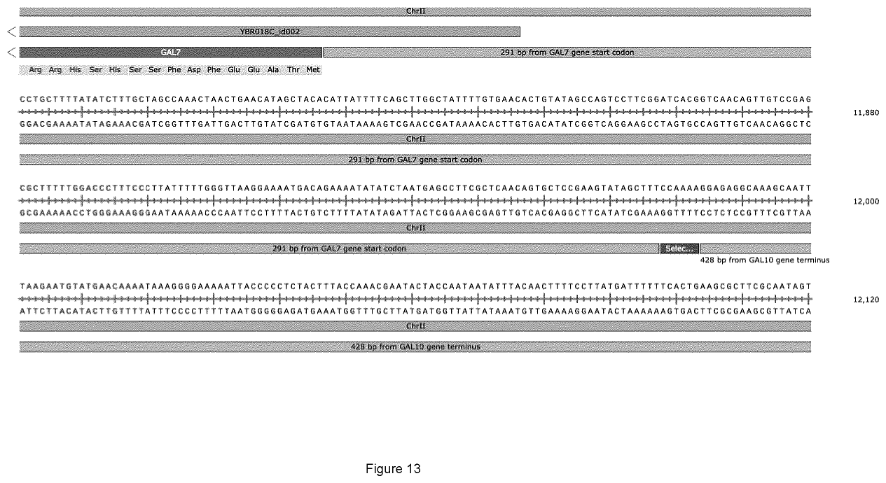

FIG. 13 illustrates a selected site for cassette integration, located in the intergenic region of two galactose utilization genes GAL10 and GAL7 at chromosome II of Saccharomyces cerevisiae. The selected site (SEQ ID NO: 34) is positioned 291 bp downstream of the gene GAL7 (SEQ ID NO: 33) and 428 bp upstream of the gene GAL10 (SEQ ID NO: 35). The inert cassette described in FIG. 12, was integrated in the site noted in FIG. 13. Integration was confirmed with Sanger sequencing and whole genome sequencing.

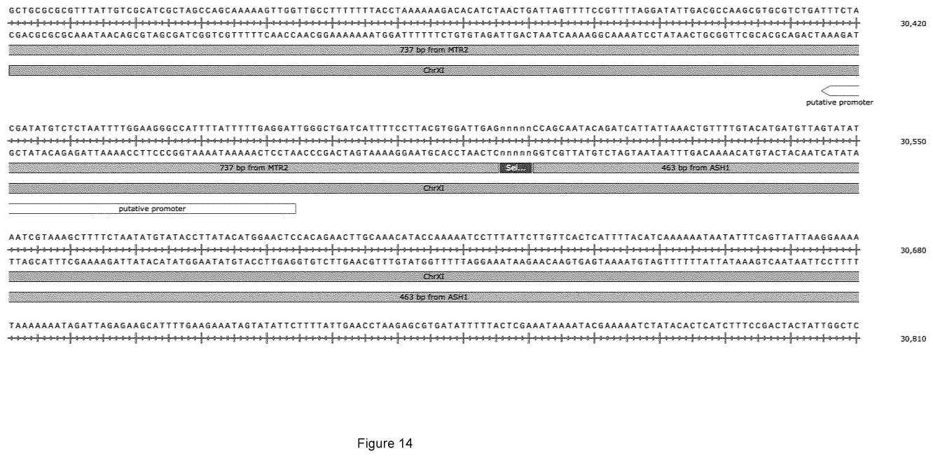

FIG. 14 illustrates a selected site for cassette integration, located in the intergenic region of the genes MTR2 and ASHI at chromosome XI of Saccharomyces cerevisiae. The selected site (SEQ ID NO: 37) is positioned 737 bp downstream of the gene MTR2 (SEQ ID NO: 36) and 463 bp upstream of the gene ASHI (SEQ ID NO: 38). The inert cassette described in FIG. 12, was integrated in the site noted in FIG. 14. Integration was confirmed with Sanger sequencing and whole genome sequencing.

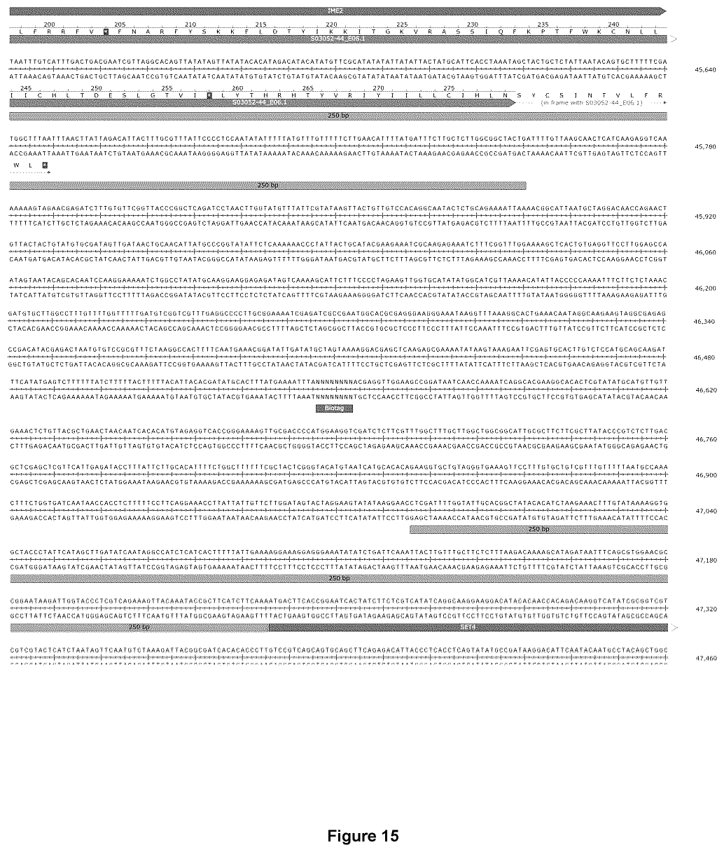

FIG. 15 illustrates a selected site located in the intergenic region of the genes IME2 and SET4 at chromosome X of Saccharomyces cerevisiae. The selected site (SEQ ID NO: 40) is positioned 1045 bp downstream of the gene IME2 (SEQ ID NO: 39) and 682 bp upstream of the gene SET4 (SEQ ID NO: 41). The inert cassette described in FIG. 12, was integrated in the site noted in FIG. 15. Integration was confirmed with Sanger sequencing and whole genome sequencing.

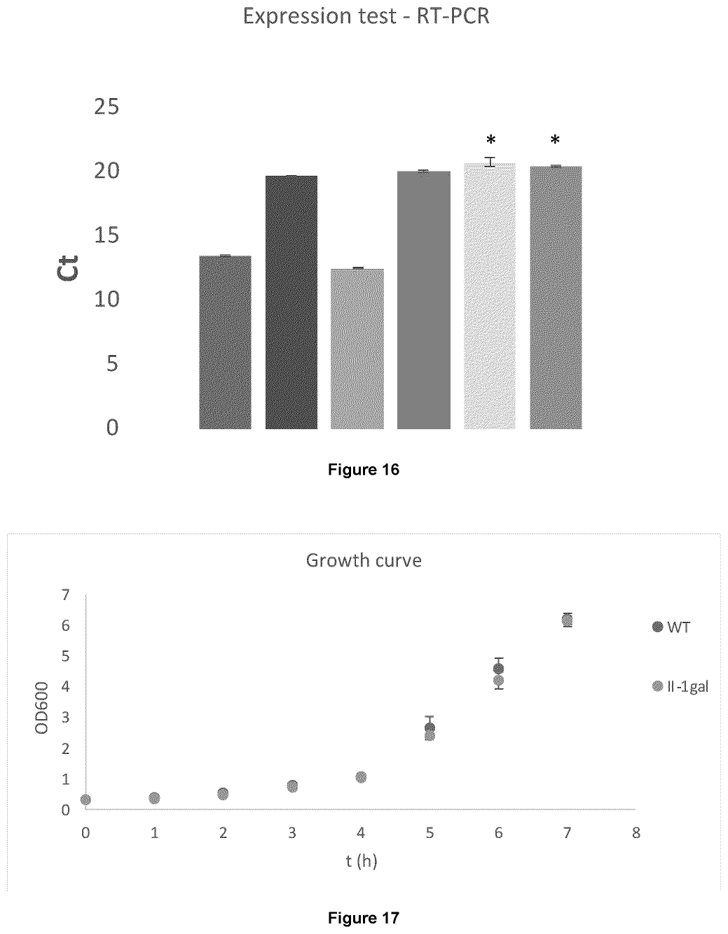

In the example depicted in FIG. 16, a Saccharomyces cerevisiae strain engineered with the inert cassette illustrated in FIG. 12 at the selected site depicted in FIG. 13, was cultured in the biomass production conditions and its RNA was extracted. The RNA was reverse transcribed to cDNA, which then was analyzed via qPCR using primers and probes that bind within the cassette. The integrant shows no statistically significant increase in the expression of the cassette (cDNA; 5th bar from the left) compared to the no-RT control (NRT; 6th bar from the left). Internal controls were used for the genes ACTI (cDNA; 1st bar from the left, NRT; 2nd bar from the left) and FBA (cDNA; 3rd bar from the left, NRT; 4th bar from the left). This shows RNA expression did not occur for this engineering event.

FIG. 17 illustrates the growth curves of a wild type Saccharomyces cerevisiae strain (blue) compared to the Saccharomyces cerevisiae strain engineered with the inert cassette illustrated in FIG. 12 at the selected site depicted in FIG. 13 (orange). In this case, the curves overlap, as measured by the OD600 over time. This indicates that the introduction of the cassette did not introduce any characteristics that affect growth with statistical significance.

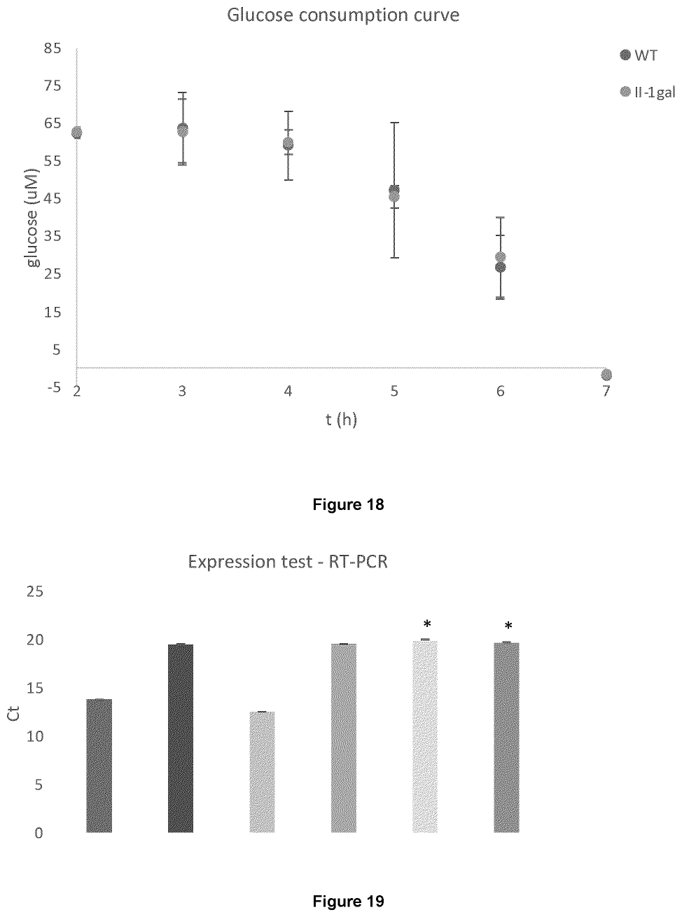

In the example depicted in FIG. 18, the sugar consumption curves of the wild type Saccharomyces cerevisiae strain (blue), is compared to the Saccharomyces cerevisiae strain engineered with the inert cassette illustrated in FIG. 12 at the selected site depicted in FIG. 13 (orange). In this case, the curves of sugar consumption overlap, as measured by Amplex red glucose assay. This indicates that the introduction of the cassette did not introduce any characteristics that affect sugar metabolism with statistical significance.

In the example depicted in FIG. 19, a Saccharomyces cerevisiae strain engineered with the inert cassette illustrated in FIG. 12 at the selected site depicted in FIG. 14, was cultured in the biomass production conditions and its RNA was extracted. The RNA was reverse transcribed to cDNA, which then was submitted to qPCR using primers and probes that bind within the cassette. The integrant shows no significant increase in the expression of the cassette (cDNA; 5th bar from the left) compared to the no-RT control (NRT; 6th bar from the left). Internal controls were used for the genes ACTI (cDNA; 1st bar from the left, NRT; 2nd bar from the left) and FBA (cDNA; 3rd bar from the left, NRT; 4th bar from the left). This shows RNA expression did not occur for this engineering event.

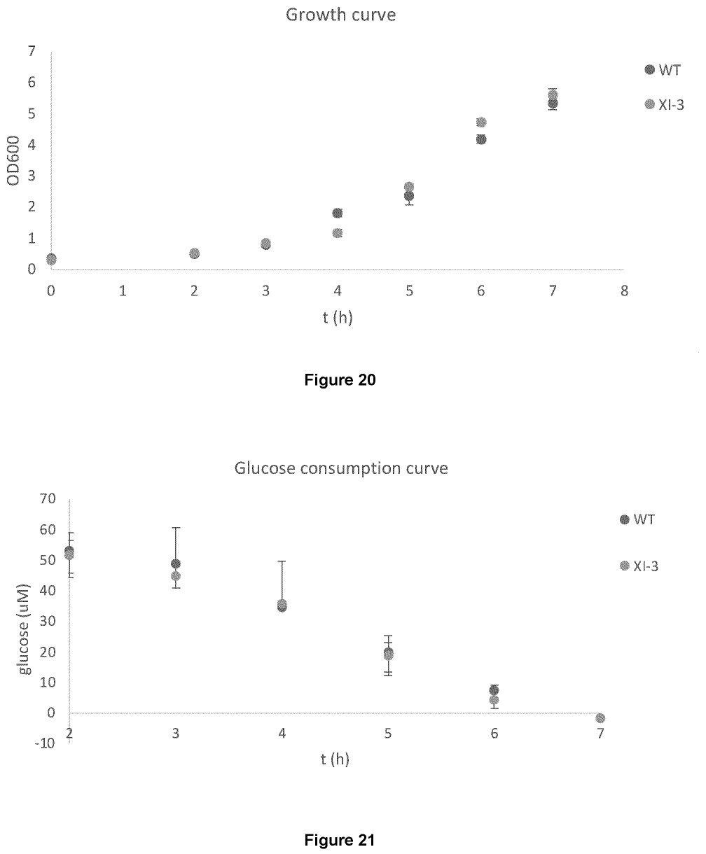

In the example depicted in FIG. 20, the growth curves of the wild type Saccharomyces cerevisiae strain (blue), are compared to a Saccharomyces cerevisiae strain engineered with the inert cassette illustrated in FIG. 12 at the selected site depicted in FIG. 14 (orange). In this case, the curves overlap, as measured by the OD600 over time. This indicates that the introduction of the cassette did not introduce any characteristics that affect growth with statistical significance.

In the example depicted in FIG. 21, the sugar consumption curves of the wild type Saccharomyces cerevisiae strain (blue), and a Saccharomyces cerevisiae strain engineered with the inert cassette described in FIG. 12, at the selected site depicted in FIG. 14 (orange). In this case, the curves of sugar consumption overlap, as measured by Amplex red glucose assay. This indicates that the introduction of the cassette did not introduce any characteristics that affect sugar metabolism with statistical significance.

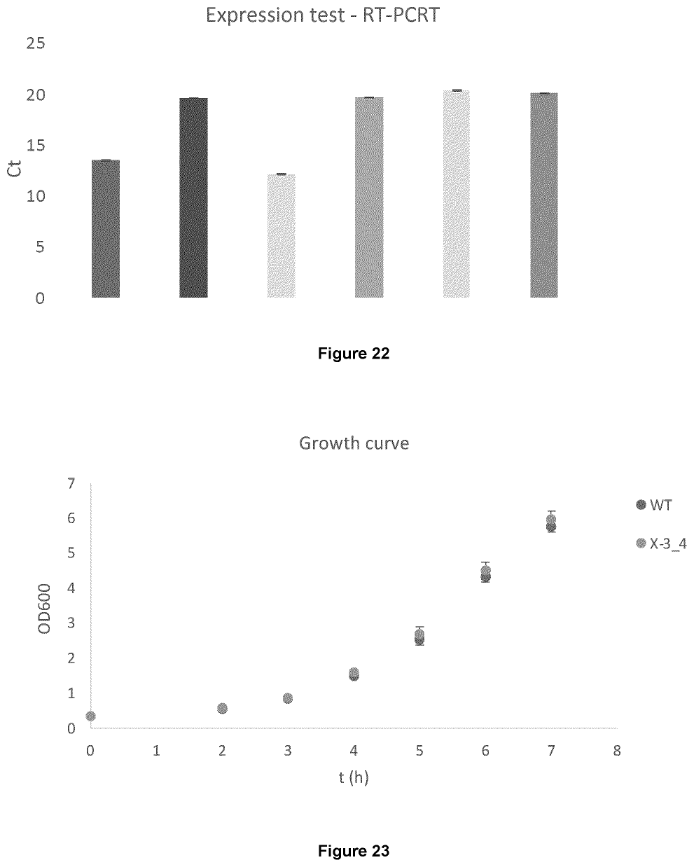

In the example depicted in FIG. 22, a Saccharomyces cerevisiae strain engineered with the inert cassette illustrated in FIG. 12, at the selected site depicted in FIG. 15, was cultured in the biomass production conditions and its RNA was extracted. The RNA was reverse transcribed to cDNA, which then was analyzed via qPCR using primers and probes that bind within the cassette. The integrant shows no statistically significant increase in the expression of the cassette (cDNA; 5th bar from the left) compared to the no-RT control (NRT; 6th bar from the left). Internal controls were used for the genes ACTI (cDNA; 1st bar from the left, NRT; 2nd bar from the left) and FBA (cDNA; 3rd bar from the left, NRT; 4th bar from the left). This shows RNA expression did not occur for this engineering event.

In the example depicted in FIG. 23, the growth curves of the wild type Saccharomyces cerevisiae strain (blue), and the Saccharomyces cerevisiae strain engineered with the inert cassette illustrated in FIG. 12 at the selected site depicted in FIG. 15 (orange) are shown. In this case, the curves overlap, as measured by the OD600 over time. This indicates that the introduction of the cassette did not introduce any characteristics that affect growth with statistical significance.

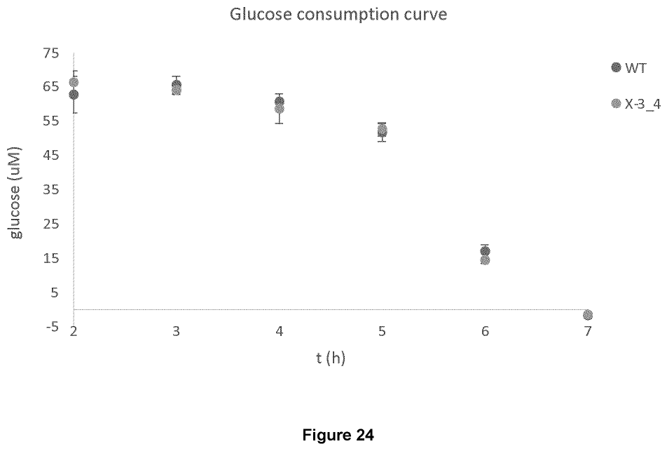

In the example depicted in FIG. 24, the sugar consumption curves of the wild type Saccharomyces cerevisiae strain (blue), and a Saccharomyces cerevisiae strain engineered with the inert cassette illustrated in FIG. 12, at the selected site depicted in FIG. 15 (orange) are shown. In this case, the curves of sugar consumption overlap, as measured by Amplex red glucose assay. This indicates that the introduction of the cassette did not introduce any characteristics that affect sugar metabolism with statistical significance.

While illustrative and presently preferred embodiments of the invention have been described in detail hereinabove, it is to be understood that the inventive concepts may be otherwise variously embodied and employed and that the appended claims are intended to be construed to include such variations except insofar as limited by the prior art.

### REFERENCES

Bourgeois, L., Pyne, M. E., & Martin, V. J. J. (2018). A Highly Characterized Synthetic Landing Pad System for Precise Multicopy Gene Integration in Yeast. ACS Synthetic Biology, 7(11), 2675-2685. https://doi.org/10.1021/acssynbio.8b00339
Bustin, S., & Huggett, J. (2017). qPCR primer design revisited. Biomolecular Detection and Quantification, 14, 19-28. https://doi.org/10.1016/j.bdq.2017.11.001
Coffin J M, Hughes S H, Varmus H E, editors. Cold Spring Harbor (NY): Cold Spring Harbor Laboratory Press; 1997.
Couto, J. A., Neves, F., Campos, F., & Hogg, T. (2005). Thermal inactivation of the wine spoilage yeasts Dekkera/Brettanomyces. International Journal of Food Microbiology, 104(3), 337-344. https://doi.org/10.1016/j.ijfoodmicro.2005.03.014
De Clerck, J. A textbook of Brewing, Translated by Kathleen Barton-Wright, Chapman & Hall, London 1958 Vol. II p 427
DiCarlo, J. E., Norville, J. E., Mali, P., Rios, X., Aach, J., & Church, G. M. (2013). Genome engineering in Saccharomyces cerevisiae using CRISPR-Cas systems. Nucleic Acids Research, 41(7), 4336-4343. https://doi.org/10.1093/nar/gkt135
Dieffenbach, C. W., Lowe, T. M., & Dveksler, G. S. (1993). General concepts for PCR primer design. Genome Research, 3(3), S30-S37. https://doi.org/10.1101/gr.3.3.530
Dujon B, Alexandraki D, André B, Ansorge W, Baladron V, Ballesta J P G, Banrevi A, Bolle P A, Bolotin-Fukuhara M, Bossier P, et al. Complete DNA sequence of yeast chromosome XI. Nature. 1994; 369:371.
Freeman, W. M., Walker, S. J., & Vrana, K. E. (1999). Quantitative RT-PCR: Pitfalls and Potential. BioTechniques, 26(1), 112-125. https://doi.org/10.2144/99261rv01
Gartenberg, M. R., & Smith, J. S. (2016). The Nuts and Bolts of Transcriptionally Silent Chromatin in Saccharomyces cerevisiae. Genetics, 203(4), 1563-1599. https://doi.org/10.1534/genetics.112.145243
Goñi, J. R., Perez, A., Torrents, D., & Orozco, M. (2007). Determining promoter location based on DNA structure first-principles calculations. Genome Biology, 8(12), R263. https://doi.org/10.1186/gb-2007-8-12-r263
Higgins, M., Ravenhall, M., Ward, D., Phelan, J., Ibrahim, A., Forrest, M. S., Clark, T. G., & Campino, S. (2018). PrimedRPA: primer design for recombinase polymerase amplification assays. Bioinformatics, 35(4), 682-684. https://doi.org/10.1093/bioinformatics/bty701
Jia, B., Li, X., Liu, W., Lu, C., Lu, X., Ma, L., Li, Y.-Y., & Wei, C. (2019). GLAPD: Whole Genome Based LAMP Primer Design for a Set of Target Genomes. Frontiers in Microbiology, 10. https://doi.org/10.3389/fmicb.2019.02860
Kucsera, J., Yarita, K., & Takeo, K. (2000). Simple detection method for distinguishing dead and living yeast colonies. Journal of Microbiological Methods, 41(1), 19-21. https://doi.org/10.1016/s0167-7012(00)00136-6
Li, Y., & Wu, C. (2013). Enhanced inactivation of Salmonella Typhimurium from blueberries by combinations of sodium dodecyl sulfate with organic acids or hydrogen peroxide. Food Research International, 54(2), 1553-1559. https://doi.org/10.1016/j.foodres.2013.09.012
Mignone, F., Gissi, C., Liuni, S., & Pesole, G. (2002). Genome Biology, 3(3), reviews0004.1. https://doi.org/10.1186/gb-2002-3-3-reviews0004
Reese, M. G. (2001). Application of a time-delay neural network to promoter annotation in the Drosophila melanogaster genome. Computers & Chemistry, 26(1), 51-56. https://doi.org/10.1016/s0097-8485(01)00099-7
Rodríguez-López, M., Cotobal, C., Fernández-Sánchez, O., Borbarán Bravo, N., Oktriani, R., Abendroth, H., Uka, D., Hoti, M., Wang, J., Zaratiegui, M., & Bähler, J. (2017). A CRISPR/Cas9-based method and primer design tool for seamless genome editing in fission yeast. Wellcome Open Research, 1, 19. https://doi.org/10.12688/wellcomeopenres.10038.3
Sheu, D.-S., Wang, Y.-T., & Lee, C.-Y. (2000). Rapid detection of polyhydroxyalkanoate-accumulating bacteria isolated from the environment by colony PCR. Microbiology, 146(8), 2019-2025. https://doi.org/10.1099/00221287-146-8-2019
Shi, S., Liang, Y., Zhang, M. M., Ang, E. L., & Zhao, H. (2016). A highly efficient single-step, markerless strategy for multi-copy chromosomal integration of large biochemical pathways in Saccharomyces cerevisiae. Metabolic Engineering, 33, 19-27. https://doi.org/10.1016/j.ymben.2015.10.011
Sonderegger, M., & Sauer, U. (2003). Evolutionary Engineering of Saccharomyces cerevisiae for Anaerobic Growth on Xylose. Applied and Environmental Microbiology, 69(4), 1990-1998. https://doi.org/10.1128/aem.69.4.1990-1998.2003
White, S. H. (1994). The evolution of proteins from random amino acid sequences: II. Evidence from the statistical distributions of the lengths of modern protein sequences. Journal of Molecular Evolution, 38(4), 383-394. https://doi.org/10.1007/bf00163155

1. A method for bioengineering a biological entity by introducing an inert nucleic acid cassette without introducing or modifying characteristics in the biological entity, the method comprising:
receiving or providing a sample comprising the biological entity having a nucleic acid sequence;
selecting an integration site in the nucleic acid sequence for inserting the inert nucleic acid cassette;
designing the inert cassette with optimized primer sequences, optimized probe sequences, optimized stop codons and disrupted start codons, and inserting the inert nucleic acid cassette into the biological entity at the integration site; and
validating that no characteristics have been added or modified in the biological entity.

2. The method according to claim 1, wherein selecting an integration site comprises determining if a new site is required.

3. The method according to claim 2, wherein determining if a new site is required comprises consulting at least one database to verify if a prior engineering event has occurred in the biological entity.

4. The method according to claim 1, wherein selecting an integration site comprises screening the genome of the biological entity and identifying any characterized putative genes, open reading frames and/or features of concern.

5. The method according to claim 4, wherein the features of concern comprises putative promoters, enhancers and any other known regulatory sequences.

6. The method according to claim 1, wherein the selected integration site is within a predetermined distance in base pairs (bp) from putative genes, open reading frames and/or features of concern.

7. The method according to claim 6, wherein the predetermined distance is between about 250 bp and about 1 kilobase pairs (kb).

8. The method according to claim 7, wherein the predetermined distance is about 250 bp.

9. The method according to claim 1, wherein the selected integration site is located within heterochromatic regions and/or long terminal repeats (LTRs).

10. The method according to claim 1, wherein selecting an integration site comprises determining an absence of characterized and/or putative promoters directing transcription of the cassette.

11. The method according to claim 1, wherein designing the inert cassette comprises creating landing pads for assisting with the integration of the inert cassette at the integration site.

12. The method according to claim 11, wherein the landing pad is a CRISPR target sequence complementary to an optimized gRNA spacer.

13. The method according to claim 1, wherein the integration site is selected in silico using at least one database.

14. The method according to claim 1, further comprising inactivating the biological entity.

15. The method according to claim 14, wherein inactivating the biological entity comprises thermal inactivation, chemical inactivation, nutrient deprivation over time or combinations thereof.

16. The method according to claim 1, wherein validating that no characteristics have been added or modified in the biological entity comprises analyzing the transcriptional profile of the biological entity to validate that transcripts of the inert nucleic acid cassette cannot be detected above a predetermined threshold.

17. The method according to claim 1, wherein validating that no characteristics have been added or modified in the biological entity comprises sequencing the whole genome of the biological entity to validate that the inert nucleic acid cassette has been integrated at the selected site, without any off-target effects.

18. The method according to claim 17, wherein the off-target effects comprises mutations in the cassette or elsewhere in the genome of the biological entity.

19. An inert nucleic acid cassette for identifying a biological material, the inert nucleic acid cassette comprising optimized primer sequences, optimized probe sequences, optimized stop codons and disrupted start codons.

20. The inert nucleic acid cassette of claim 19, wherein the optimized stop codons comprises stop codons for all reading frames in order to stop any transcription initiation within the cassette and the sequences flanking the cassette.


SEQUENCE LISTING (41 sequences)

SEQ ID NO: 1 - DNA, 313 bp, Artificial Sequence
  complete sequence of an inert cassette
ttaattaattatttatatatttaaatttaggacactccccccacttcattcaggctgcgcaactgaggctgagggtggacctgatcaacctgaaccaggaccccagtttaattaattaacccccacgagagcgagttccccgacactcggccaggtgcccaccgtggtgagcctggagcacttccccaagggccacgcctacaccgccctggagtgcgggcctcttcgctattacgccagctggcgaaagggggatctgctgggctagttaattgctcagcggttaattatattatatttatatattaattaa

SEQ ID NO: 2 - DNA, 25 bp, Artificial Sequence
  stop codons
ttaattaattatttatatatttaaa

SEQ ID NO: 3 - DNA, 22 bp, Artificial Sequence
  qPCR primer forward (Top strand)
tttaggacactccccccacttc

SEQ ID NO: 4 - DNA, 19 bp, Artificial Sequence
  LAMP F3
actccccccacttcattca

SEQ ID NO: 5 - DNA, 18 bp, Artificial Sequence
  LAMP FIP 2
gctgagggtggacctgat

SEQ ID NO: 6 - DNA, 12 bp, Artificial Sequence
  optimal stop codon configuration
aattaattaatt

SEQ ID NO: 7 - DNA, 20 bp, Artificial Sequence
  FIP 1
cacgagagcgagttccccga

SEQ ID NO: 8 - DNA, 23 bp, Artificial Sequence
  Probe qPCR
tcggccaggtgcccaccgtggtg

SEQ ID NO: 9 - DNA, 20 bp, Artificial Sequence
  BIP 1
gtggtgagcctggagcactt

SEQ ID NO: 10 - DNA, 20 bp, Artificial Sequence
  BIP 2
cctcttcgctattacgccag

SEQ ID NO: 11 - DNA, 18 bp, Artificial Sequence
  LAMP B3
cgaaagggggatctgctg

SEQ ID NO: 12 - DNA, 21 bp, Artificial Sequence
  qPCR primer reverse (5′ - 3′)
ccgctgagcaattaactagcc

SEQ ID NO: 13 - DNA, 30 bp, Artificial Sequence
  stop codons
ttaattatattatatttatatattaattaa

SEQ ID NO: 14 - DNA, 322 bp, Artificial Sequence
  a complete sequence of an inert cassette
ttaattaattaaaagctttagtggcccccctaatcaagttccgctatccagagtcgtcgtgagcacgactgtctctcccagcgaatcgctacgagcaatagaccgcggactcgcgaacgaacaatactaagggggccttagcgcgttaaaggttaattaattaactggaagcttcgccagttattccccctatcgaaagagtcgcagccacaattgcctatttcgtgaaagggatcagagccctgatcggaatcgatacctcgcctgtacgggtcaatacggaaaattccaattgttgcgacaaggccggttaattaattaa

SEQ ID NO: 15 - DNA, 12 bp, Artificial Sequence
  stop codons
ttaattaattaa

SEQ ID NO: 16 - DNA, 4 bp, Artificial Sequence
  PAM site
ttta

SEQ ID NO: 17 - DNA, 21 bp, Artificial Sequence
  gRNA binding site
gtggcccccctaatcaagttc

SEQ ID NO: 18 - DNA, 20 bp, Artificial Sequence
  sequencing primer forward
gcacgactgtctctcccagc

SEQ ID NO: 19 - DNA, 18 bp, Artificial Sequence
  F3
gcaatagaccgcggactc

SEQ ID NO: 20 - DNA, 30 bp, Artificial Sequence
  RPA_F
ccgcggactcgcgaacgaacaatactaagg

SEQ ID NO: 21 - DNA, 20 bp, Artificial Sequence
  qPCR_primer_F
ggactcgcgaacgaacaata

SEQ ID NO: 22 - DNA, 18 bp, Artificial Sequence
  F2
actaagggggccttagcg

SEQ ID NO: 23 - DNA, 12 bp, Artificial Sequence
  stop codons
ttaattaattaa

SEQ ID NO: 24 - DNA, 25 bp, Artificial Sequence
  Probe_qPCR
actggaagcttcgccagttattccc

SEQ ID NO: 25 - DNA, 22 bp, Artificial Sequence
  F1c
ccccctatcgaaagagtcgcag

SEQ ID NO: 26 - DNA, 30 bp, Artificial Sequence
  RPA_R (5′ - 3′)
tttcacgaaataggcaattgtggctgcgac

SEQ ID NO: 27 - DNA, 19 bp, Artificial Sequence
  qPCR_primer_R (5′ - 3′)
attgtggctgcgactcttt

SEQ ID NO: 28 - DNA, 22 bp, Artificial Sequence
  B1c
ttcgtgaaagggatcagagccc

SEQ ID NO: 29 - DNA, 20 bp, Artificial Sequence
  sequencing primer_R (5′ - 3′)
ttgacccgtacaggcgaggt

SEQ ID NO: 30 - DNA, 20 bp, Artificial Sequence
  B2
cctgtacgggtcaatacgga

SEQ ID NO: 31 - DNA, 19 bp, Artificial Sequence
  B3
ccaattgttgcgacaaggc

SEQ ID NO: 32 - DNA, 12 bp, Artificial Sequence
  stop codons
ttaattaattaa

SEQ ID NO: 33 - DNA, 291 bp, Saccharomyces cerevisiae
ttttgagggaatattcaactgtttttttttatcatgttgatgctctgcataataatgcccataaatatttccgacctgcttttatatctttgctagccaaactaactgaacatagctacacattattttcagcttggctattttgtgaacactgtatagccagtccttcggatcacggtcaacagttgtccgagcgctttttggaccctttcccttatttttgggttaaggaaaatgacagaaaatatatctaatgagccttcgctcaacagtgctccgaagtatagcttt

SEQ ID NO: 34 - DNA, 6 bp, Saccharomyces cerevisiae
ccaaaa

SEQ ID NO: 35 - DNA, 137 bp, Saccharomyces cerevisiae
ggagaggcaaagcaatttaagaatgtatgaacaaaataaaggggaaaaattaccccctctactttaccaaacgaatactaccaataatatttacaacttttccttatgattttttcactgaagcgcttcgcaatagt

SEQ ID NO: 36 - DNA, 177 bp, Saccharomyces cerevisiae
gccagcaaaaagttggttgcctttttttacctaaaaaagacacatctaactgattagttttccgttttaggatattgacgccaagcgtgcgtctgatttctacgatatgtctctaattttggaagggccattttatttttgaggattgggctgatcattttccttacgtggattgag

SEQ ID NO: 37 - DNA, 24 bp, Saccharomyces cerevisiae
ttacgtggattgagccagcaatac

SEQ ID NO: 38 - DNA, 310 bp, Saccharomyces cerevisiae
ccagcaatacagatcattattaaactgttttgtacatgatgttagtatataatcgtaaagcttttctaatatgtataccttatacatggaactccacagaacttgcaaacataccaaaaatcctttattcttgttcactcattttacatcaaaaaataatatttcagttattaaggaaaataaaaaaatagattagagaagcattttgaagaaatagtatattcttttattgaacctaagagcgtgatatttttactcgaaataaaatacgaaaaatctatacactcatctttccgactactattggctc

SEQ ID NO: 39 - DNA, 250 bp, Saccharomyces cerevisiae
taatttgtcatttgactgacgaatcgttaggcacagttatatagttatatacacatagacatacatatgttcgcatatatattatattactatgcattcacctaaatagctactgctctattaatacagtgctttttcgatggctttaatttaacttattagacattactttgcgtttattcccctccaatatatttttatgtttgtttttcttgaacattttatgatttcttgctcttggcggctactg

SEQ ID NO: 40 - DNA, 24 bp, Saccharomyces cerevisiae
ctttatgaaaatttaacgaggttg

SEQ ID NO: 41 - DNA, 250 bp, Saccharomyces cerevisiae
ctcgattttggtattgcacggctatacacatctaagaaactttgtataaaaggtggctaccctattcatagcttgatatcaataggccatctcatcactttttattgaaaaggaaaggagggaaatatatctgattcaaattacttgtttgcttctctttaagacaaaagcatagataatttcagcgtggaacgccggaataagattggtaccctcgtcagaaagttacaaataccgcttcatcttcaaa